(19) United States
(12) Patent Application Publication
(10) Pub. No.: US 2018/0149680 A1
(43) Pub. Date: May 31, 2018

(54) VOLTAGE BUFFER FOR CURRENT SENSOR

(71) Applicant: QUALCOMM Incorporated, San Diego, CA (US)

(72) Inventors: Bo SUN, Carlsbad, CA (US); Joung Won PARK, San Diego, CA (US)

(21) Appl. No.: 15/362,762

(22) Filed: Nov. 28, 2016

(51) Int. Cl. G01R 19/00; G01R 15/16; C12Q 1/68

(52) U.S. Cl. CPC G01R 19/0061; C12Q 1/6869; G01R 15/16; G01R 19/0092

(57) ABSTRACT

A sensing cell includes a current sensor, an integrating capacitor, and a voltage buffer. The integrating capacitor is configured to store a voltage representative of a current signal generated by the current sensor. The voltage buffer is coupled to provide a buffered voltage to a readout line and includes a first transistor and a second transistor. The first transistor is coupled to receive the voltage stored on the integrating capacitor and the second transistor is coupled to the readout line. The second transistor is configured to compensate for a body effect of the first transistor.

### FIELD OF DISCLOSURE

This disclosure relates generally to current sensors, and in particular, but not exclusively to electronic circuits for the voltage buffer of an ionic current sensor.

### BACKGROUND

Advances in micro-miniaturization within the semiconductor industry in recent years have enabled biotechnologists to begin packing traditionally bulky sensing tools into smaller and smaller form factors, onto so-called biochips. Despite these advances in micro-miniaturization there remains a need to (1) further miniaturize the chips, (2) to increase their throughput, and/or (2) to improve their performance For example, for many current sensors, such as the current sensors utilized in biochips, the sensed current is typically a very small signal. Thus, a low-noise front-end design may be important in maintaining the accuracy and/or reliability of the sensor.

### SUMMARY

The following presents a simplified summary relating to one or more aspects and/or embodiments associated with the mechanisms disclosed herein for a voltage buffer of a current sensor. As such, the following summary should not be considered an extensive overview relating to all contemplated aspects and/or embodiments, nor should the following summary be regarded to identify key or critical elements relating to all contemplated aspects and/or embodiments or to delineate the scope associated with any particular aspect and/or embodiment. Accordingly, the following summary presents certain concepts relating to one or more aspects and/or embodiments relating to the mechanisms disclosed herein to buffer a voltage of a current sensor in a simplified form to precede the detailed description presented below.

According to one aspect, a sensing cell includes a current sensor, an integrating capacitor, and a voltage buffer. The integrating capacitor is configured to store a voltage representative of a current signal generated by the current sensor. The voltage buffer is coupled to provide a buffered voltage to a readout line and includes a first transistor and a second transistor. The first transistor is coupled to receive the voltage stored on the integrating capacitor and the second transistor is coupled to the readout line. The second transistor is configured to compensate for a body effect of the first transistor.

According to another aspect, a sensing cell includes means for generating a current signal, means for storing a voltage representative of the current signal, and a voltage buffer. The voltage buffer is coupled to the means for storing the voltage and is configured to generate a buffered voltage. The voltage buffer includes a first transistor and a second transistor. The first transistor is coupled to receive the voltage representative of the current signal and the second transistor is coupled to generate the buffered voltage and is configured to compensate for a body effect of the first transistor.

According to yet another aspect, a complementary metal-oxide-semiconductor (CMOS) ionic current sensor array includes a plurality of sensing cells, where each sensing cell includes an ionic current sensor, an integrating capacitor, a sense field effect transistor (FET), and a voltage buffer. The ionic current sensor is configured to generate a current signal and the integrating capacitor is configured to store a voltage representative of the current signal. The sense FET is coupled between the integrating capacitor and the current sensor to provide the current signal to the integrating capacitor and the voltage buffer is coupled between the integrating capacitor and a column readout line to provide a buffered voltage to the column readout line. The voltage buffer includes a first FET, a second FET, a first current source, and a second current source. The first FET is coupled to receive the voltage stored on the integrating capacitor and the second FET is coupled to the column readout line and also is configured to compensate for a body effect of the first FET. The first current source is coupled to the first and second FETs and the second current source is coupled to the second FET.

### BRIEF DESCRIPTION OF THE DRAWINGS

The accompanying drawings are presented to aid in the description of present disclosure and are provided solely for illustration of the embodiments and not limitation thereof.

FIG. 1 illustrates an example ionic current sensor configured for the sequencing of a single stranded DNA molecule, in accordance with an aspect of the present disclosure.

FIG. 2 is a block diagram illustrating an example current sensor array, in accordance with an aspect of the present disclosure.

FIG. 3A is a circuit diagram illustrating an example sensing cell, in accordance with an aspect of the present disclosure.

FIG. 3B is a diagram illustrating a gain performance of an example implementation of the sensing cell of FIG. 3A.

FIG. 4A is a circuit diagram illustrating another example sensing cell, in accordance with an aspect of the present disclosure.

FIG. 4B is a diagram illustrating a gain performance of an example implementation of the sensing cell of FIG. 4A.

FIG. 5A is a circuit diagram illustrating yet another example sensing cell, in accordance with an aspect of the present disclosure.

FIG. 5B is a diagram illustrating a linearity and a gain performance of an example implementation of the sensing cell of FIG. 5A.

FIG. 6A is a circuit diagram illustrating still another example sensing cell, in accordance with an aspect of the present disclosure.

FIG. 6B is a diagram illustrating a linearity and a gain performance of an example implementation of the sensing cell of FIG. 6A.

### DETAILED DESCRIPTION

Aspects of the present disclosure are disclosed in the following description and related drawings directed to specific examples. Alternate implementations may be devised without departing from the scope of the present disclosure. Additionally, well-known aspects will not be described in detail or will be omitted so as not to obscure the relevant details of the present disclosure.

The terminology used herein is for the purpose of describing particular embodiments only and is not intended to be limiting of embodiments of the present disclosure. As used herein, the singular forms “a”, “an” and “the” are intended to include the plural forms as well, unless the context clearly indicates otherwise. It will be further understood that the terms “comprises”, “comprising,”, “includes” and/or “including”, when used herein, specify the presence of stated features, integers, steps, operations, elements, and/or components, but do not preclude the presence or addition of one or more other features, integers, steps, operations, elements, components, and/or groups thereof.

Further, some embodiments may be described in terms of sequences of actions to be performed by, for example, elements of a computing device. It will be recognized that various actions described herein can be performed by specific circuits (e.g., application specific integrated circuits (ASICs)), by program instructions being executed by one or more processors, or by a combination of both. Additionally, these sequence of actions described herein can be considered to be embodied entirely within any form of computer readable storage medium having stored therein a corresponding set of computer instructions that upon execution would cause an associated processor to perform the functionality described herein. Thus, the various aspects of the present disclosure may be embodied in a number of different forms, all of which have been contemplated to be within the scope of the claimed subject matter. In addition, for each of the embodiments described herein, the corresponding form of any such embodiments may be described herein as, for example, “means for” or “logic configured to” perform the described action.

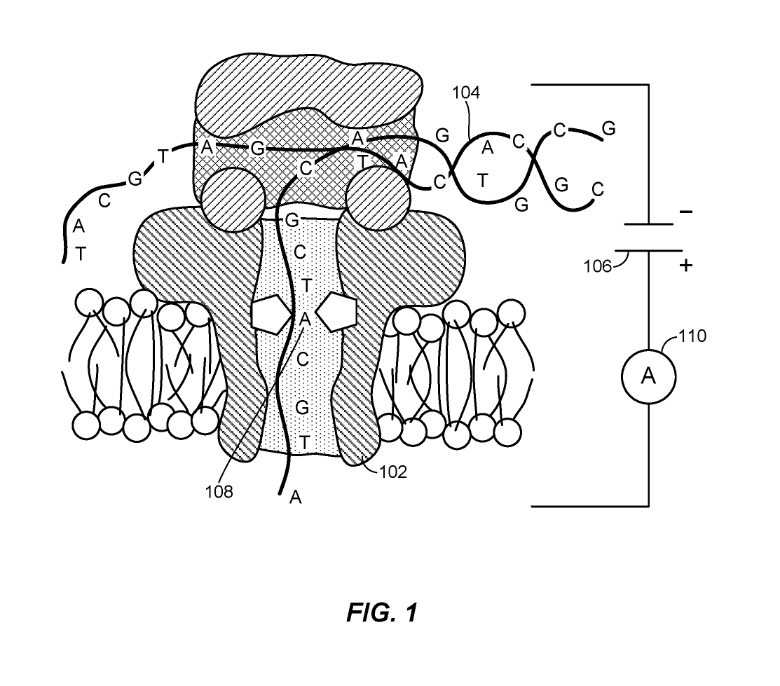

FIG. 1 illustrates an example ionic current sensor 102 configured for the sequencing of single stranded DNA (ssDNA) 104. The ionic current sensor 102 is shown as including an opening 108 (also referred to as a “pore”). When configured for rapid nucleotide sequencing, the opening 108 of ionic current sensor 102 may have an internal diameter on the order of a few (e.g., 1, 2, etc.) nanometers. Thus, in these applications, the opening 108 may also be referred to as a “nanopore”.

In operation, a voltage potential 106 is applied across the opening 108, which may be immersed in a conducting fluid. When the voltage potential 106 is applied, a small ionic current 110 attributable to the conduction of ions across the opening 108 can be sensed. The amount of ionic current 110 that is sensed is relative to the size (i.e., internal diameter of opening 108). When a molecule, such as a DNA or RNA molecule, passes through the opening 108, it can partially or completely block the opening 108, causing a change in a magnitude of the ionic current 110. It has been shown that such an ionic current blockade can be correlated with the base pair sequence of DNA or RNA molecules.

In practice, nanopore-based DNA sequencers may include a large array of complementary metal-oxide-semiconductor (CMOS) ionic current sensors, such as ionic current sensor 102. Each ionic current sensor of the array may be included in a respective sensing cell that includes electronic circuitry for controlling the operation and monitoring the output of each ionic current sensor 102. For example, each sensing cell may be configured to generate the voltage potential 106 and to sense the ionic current 110 generated by a respective ionic current sensor. In some applications the ionic current 110 is in the range of 10 pA-100 pA. In other words, the sensed current (i.e., ionic current 110) is a very small signal. Thus, a low-noise front-end design may be important in maintaining the accuracy and/or reliability of the sensor. Among front-end circuits, a good voltage buffer for generating the voltage representative of the ionic current 110 is desired which provides a gain that is close to unity and also provides a substantially linear output.

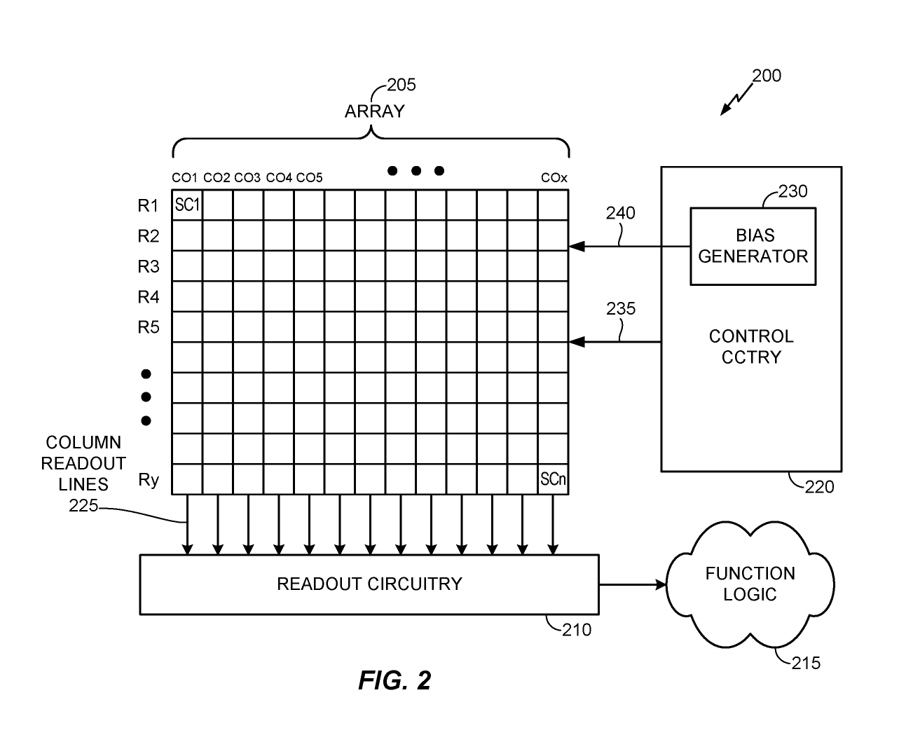

FIG. 2 is a block diagram illustrating a current sensor array 200, in accordance with an aspect of the present disclosure. The illustrated example of current sensor array 200 includes an array 205, readout circuitry 210, function logic 215, and control circuitry 220.

In one example, array 205 is a two-dimensional array of sensing cells (e.g., sensing cells SC1, . . . , SCn). Each sensing cell may be a complementary metal-oxide-semiconductor (“CMOS”) sensing cell, where each sensing cell includes at least one ionic current sensor, such as ionic current sensor 102 of FIG. 1. As illustrated, each sensing cell is arranged into a row (e.g., rows R1 to Ry) and a column (e.g., column CO1 to COx) to sense ionic current through a respective ionic current sensor. In one application, the sensed ionic current can then be used to determine the base pair sequence of DNA and/or RNA molecules.

Control circuitry 220 is coupled to array 205 to control operational characteristics of array 205 via one or more control signals 235. For example, control circuitry 220 may generate a row select, column select, and/or a reset signal for controlling acquisition of the ionic current values by the sensing cells.

As illustrated in FIG. 2, control circuitry 220 may further include a bias generator 230. bias generator 230 is configured to generate a bias voltage 240 that is provided to each sensing cell in the array 205. Each sensing cell of the array 205 may include its own amplifier that receives the bias voltage 240. Each amplifier may then generate a reference voltage for biasing the current sensor within a respective sensing cell. The reference voltage generated by each amplifier may track process and temperature variations to control the sensing speed of the current sensor array 200. The reference voltage may also be adjustable to provide an optimum sensing result. Within each sensing cell of array 205, this reference voltage can be adjusted to reduce the mismatch.

After each sensing cell has sensed its current, the current data or values are readout by readout circuitry 210 which are then transferred to function logic 215. Readout circuitry 210 may include amplification circuitry, analog-to-digital conversion circuitry, or otherwise. Function logic 215 may simply storage the current values or even analyze ionic current values to determine the DNA/RNA sequencing. In one example, readout circuitry 210 may readout a row of ionic current values at a time along column readout lines 225 or may readout the current values using a variety of other techniques (not illustrated), such as a serial readout or a full parallel readout of all sensing cells simultaneously. In one example, the current values are readout via column readout lines 225 in the form of a buffered voltage generated within each sensing cell. However, as mentioned above, the current signal sensed within each sensing cell is a very small signal. Thus, the front-end circuit including a voltage buffer within each sensing cell is designed to provide a gain that is close to unity and also to provide a substantially linear output.

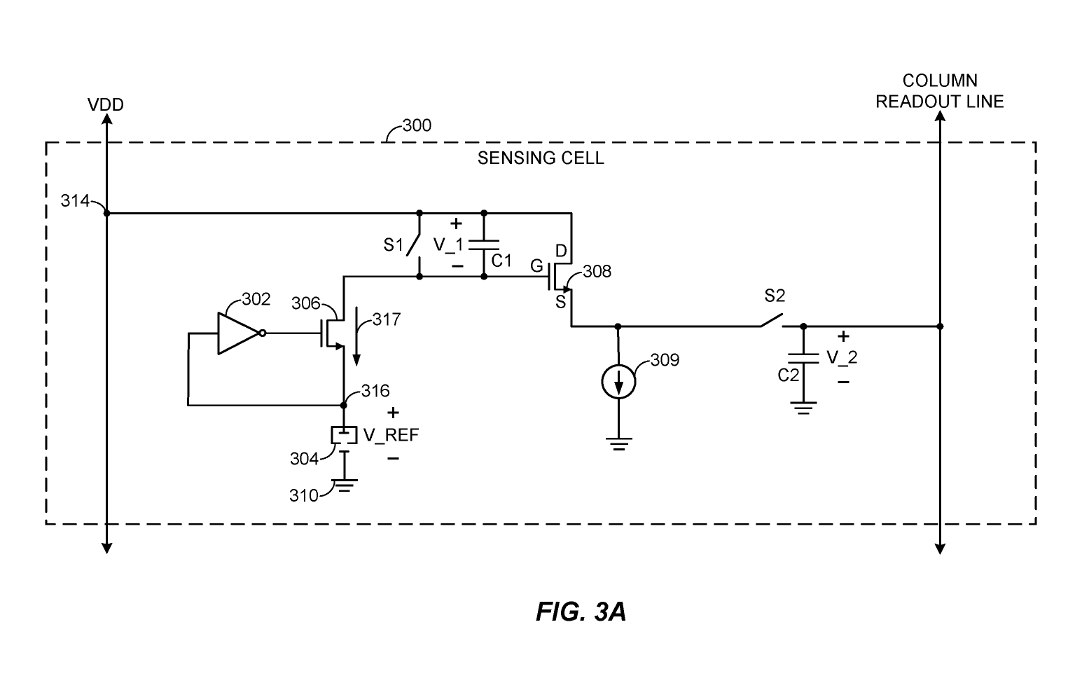

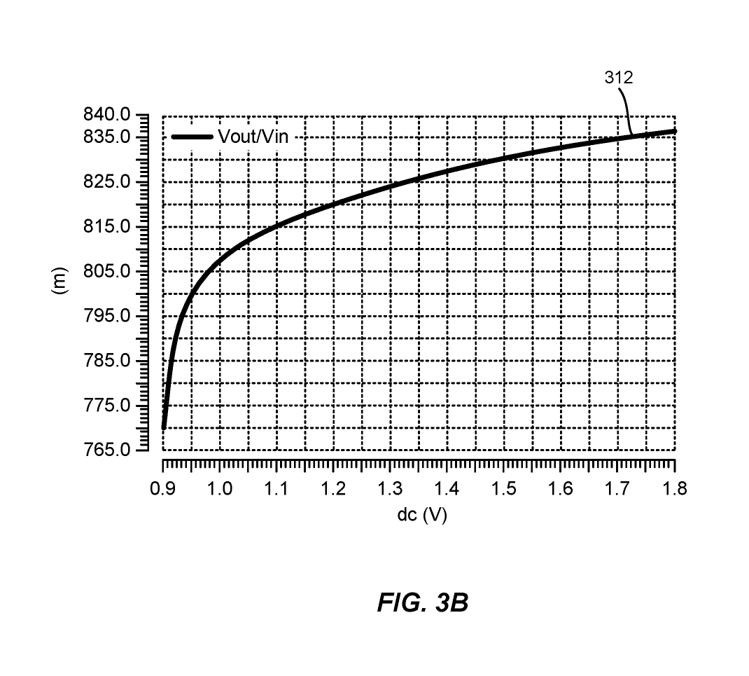

By way of example, FIG. 3A is a circuit diagram illustrating an example sensing cell 300 that implements a source follower utilized as a voltage buffer, in accordance with an aspect of the present disclosure. The illustrated example of sensing cell 300 includes an amplifier 302, a current sensor 304, a sense transistor 306, a source follower transistor 308, a current source 309, a common reference 310, a supply voltage node 314, a reference voltage node 316, integrating capacitor C1, an output capacitor C2, and switches S1 and S2. Also illustrated in FIG. 3A is a current 317, a voltage V_1, and a buffered voltage V_2.

Sensing cell 300 of FIG. 3A illustrates one possible circuitry architecture for implementing each sensing cell within array 205 of FIG. 2. Furthermore, in one example, current sensor 304 may be an ionic current sensor, such as ionic current sensor 102 of FIG. 1. However, it should be appreciated that embodiments of the present disclosure are not limited to the illustrated circuitry architectures or the referenced implementations of the illustrated circuit; rather, one of ordinary skill in the art having the benefit of the instant disclosure will understand that the present teachings are also applicable to various other circuitry architectures and implementations. By way of example, sensing cell 300 may be implemented in a general current sensor for sensing a small signal current in implementations other than the ionic current sensing implementations mentioned above.

During a readout operation of the current sensor 304, switch S1 may receive a pre-charge signal (not shown) to control switch S1 to pre-charge the integrating capacitor C1 to the supply voltage VDD. In some implementations, switch S1 may be referred to as a reset switch and may include one or more transistors. Amplifier 302 may be coupled to provide the reference voltage V_REF to bias the current sensor 304. In one example, amplifier 302 may be configured to provide the reference voltage V_REF based on a bias voltage received via a bias generator (e.g., bias voltage 240 received from bias generator 230 of FIG. 2). The sense transistor 306 may be coupled between the integrating capacitor C1 and the current sensor 304 such that the current 317 generated by the current sensor 304 may be integrated onto integrating capacitor C1 (e.g., by reducing the charge on integrated capacitor C1).

Thus, in one example, the voltage V_1 across the integrating capacitor C1 may be representative of the sensed current 317. In one example, the charge stored in the capacitor is represented by Q=CV, where Q is the charge, C is capacitance of capacitor C1 and V is the voltage V_1 across the capacitor. The charge is integration of the current over time. Thus, as current is allowed to flow through the capacitor C1, the charge and voltage across the capacitor C1 linearly increases over time. Source follower transistor 308 and current source 309 are configured as a voltage buffer between integrating capacitor C1 and the column readout line. Switch S2 is coupled between the source follower transistor 308 and the column readout line to provide the buffered voltage V_2 in response to a transfer signal (not shown). As shown in FIG. 3A, sensing cell 300 may further include an output capacitor C2 coupled between the switch S2 and the column readout line. In one aspect, output capacitor C2 functions as a sampling capacitor for subsequent analog-to-digital converter (ADC) circuitry (e.g., ADC included in readout circuitry 210 of FIG. 2). In one example, the pre-charge (i.e., reset) signal applied to switch S1 and the transfer signal applied to switch S2 are provided by way of the control signals 235 generated by control circuitry 220 of FIG. 2.

The transfer signal, the pre-charge (e.g., reset) signal, supply voltage VDD, and the common reference 310 (e.g., ground) may be routed in the sensing cell 300 by way of metal interconnect layers (i.e., routings) included in the array (e.g., array 205).

Although FIG. 3A illustrates sense transistor 306 and source follower transistor 308 as field effect transistors (FETs), other transistor types may be implemented for one or more of the illustrated transistors, including, but not limited to bipolar junction transistors (BJTs).

As mentioned above, the source follower transistor 308 and current source 309 are configured as a voltage buffer to provide the buffered voltage V_2 to the column readout line. For example, the gate G of the source follower transistor 308 is coupled to receive the voltage V_1 that is stored on the integrating capacitor C1 and in response thereto, generate the buffered voltage V_2 at the source S of the source follower transistor 308. However, the source follower transistor 308 of FIG. 3A may suffer from an effect known as the “body effect.” In one aspect, the body effect refers to a change in the transistor threshold voltage (VT) resulting from a voltage difference between the transistor source and body. Because the voltage difference between the source and body affects the VT, the body can be thought of as a second gate that helps determine how the transistor turns on and off. Thus, in the configuration of source follower transistor 308 of FIG. 3A, the gain of the voltage buffer is reduced. That is, the buffered voltage V_2/the stored voltage V_1 is less than unity (1.0) and in fact may be a non-negligible amount below unity. By way of example, FIG. 3B is a diagram illustrating a gain performance of an example implementation of the sensing cell of FIG. 3A. In one example, the gain 312 of FIG. 3B represents the gain of the voltage buffer provided by the source follower transistor 308 and current source 309 with respect to input voltage (e.g., buffered voltage V_2). In one aspect, the gain of the voltage buffer is equal to the buffered voltage V_2/the stored voltage V_1. As shown in FIG. 3B, the gain of the voltage buffer is a non-negligible amount below unity and is in the range of about 0.800-0.835. In addition to the loss of gain due to the body effect, the source follower transistor 308 of FIG. 3A may suffer from non-linearity at some of the process corners. That is, the buffered voltage V_2 provided to the column readout line may be substantially non-linear.

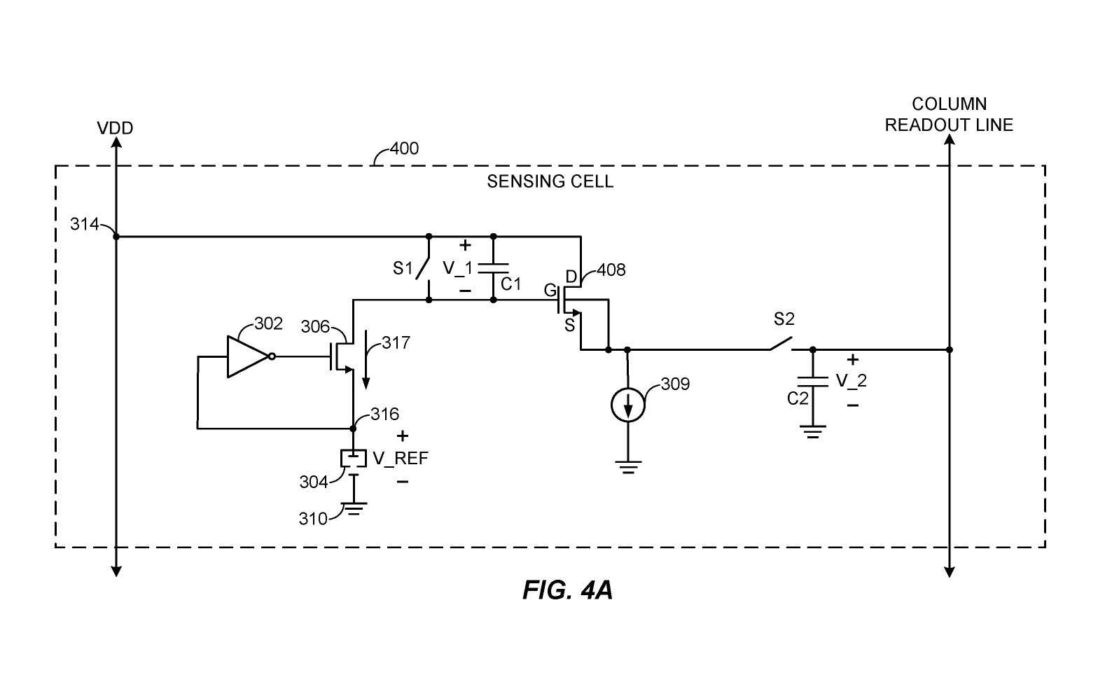

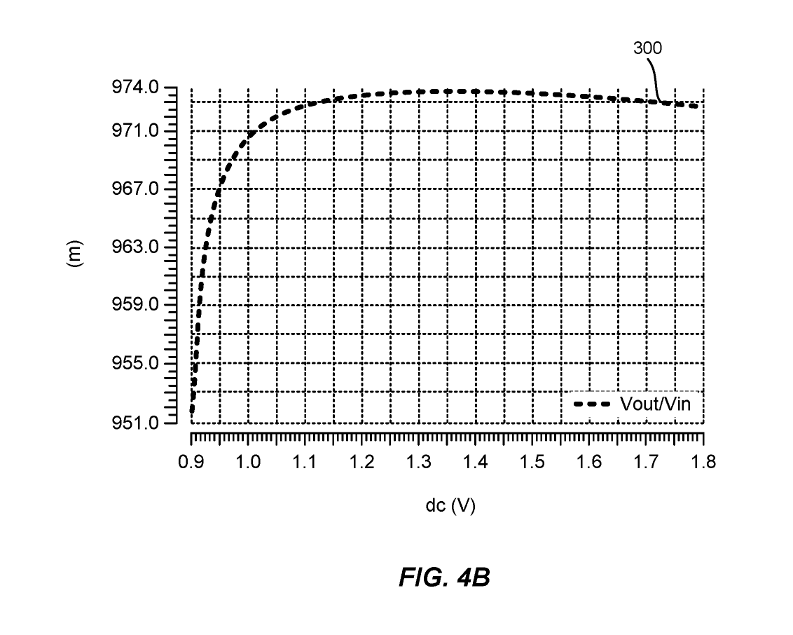

FIG. 4A is a circuit diagram illustrating another example sensing cell 400, in accordance with an aspect of the present disclosure. Sensing cell 400 is similar to sensing cell 300 of FIG. 3A, where like numerals and like labels are used to refer to like elements. However, the illustrated example of sensing cell 400 includes a voltage buffer implemented as a source follower utilizing a deep N-well (DNW) device 408. In one example, the DNW device 408 is an NMOS transistor that is fabricated in a P well or a substrate, where the NMOS transistor is completely surrounded by an N-type diffusion. In this case, a deep N well is formed by a relatively high energy ion implantation. A connection to the deep N well may be formed by an N well ring that is connected to a supply voltage, such as VDD. The DNW device may have the effect of decreasing noise and providing a much better gain and linearity for the illustrated voltage buffer. For example, FIG. 4B is a diagram illustrating a gain performance of an example implementation of the sensing cell 400 of FIG. 4A. As shown in FIG. 4B, the gain of the voltage buffer is in the range of about 0.971-0.974. However, the implications on the layout of the DNW device 408 are a larger area required for the device due to the extra N well rings used to connect to the deep N well. Thus, the DNW device 408 may improve gain and linearity, but may be difficult to implement due to processing design rules (e.g., spacing requirements for small cell sizes may make fabricating a DNW device difficult, if not practically impossible).

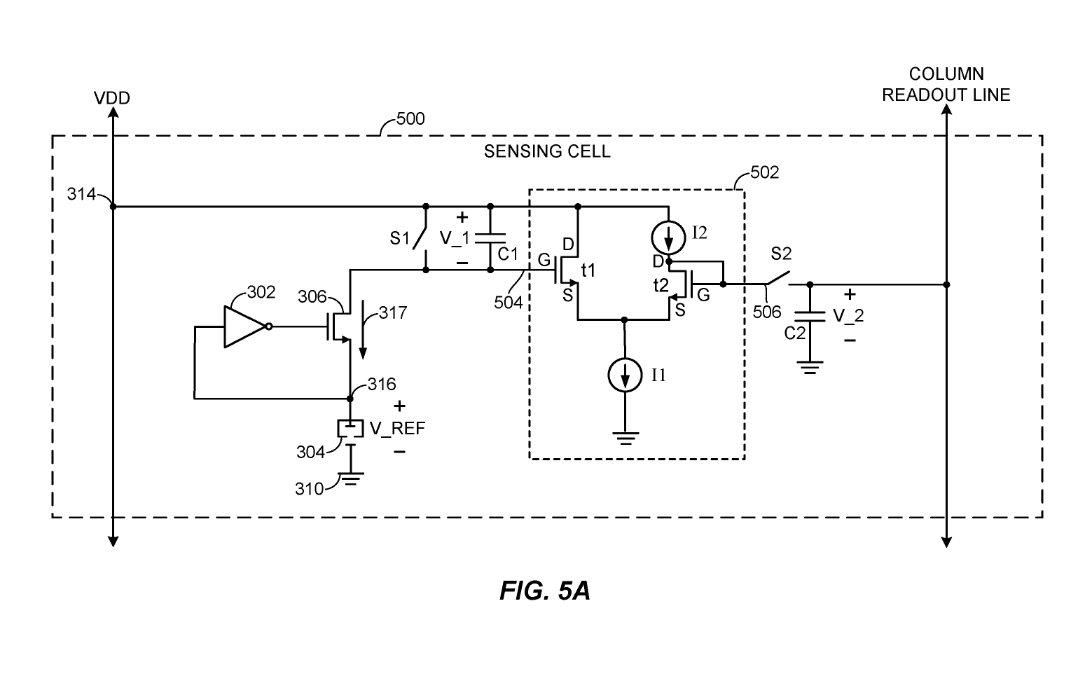

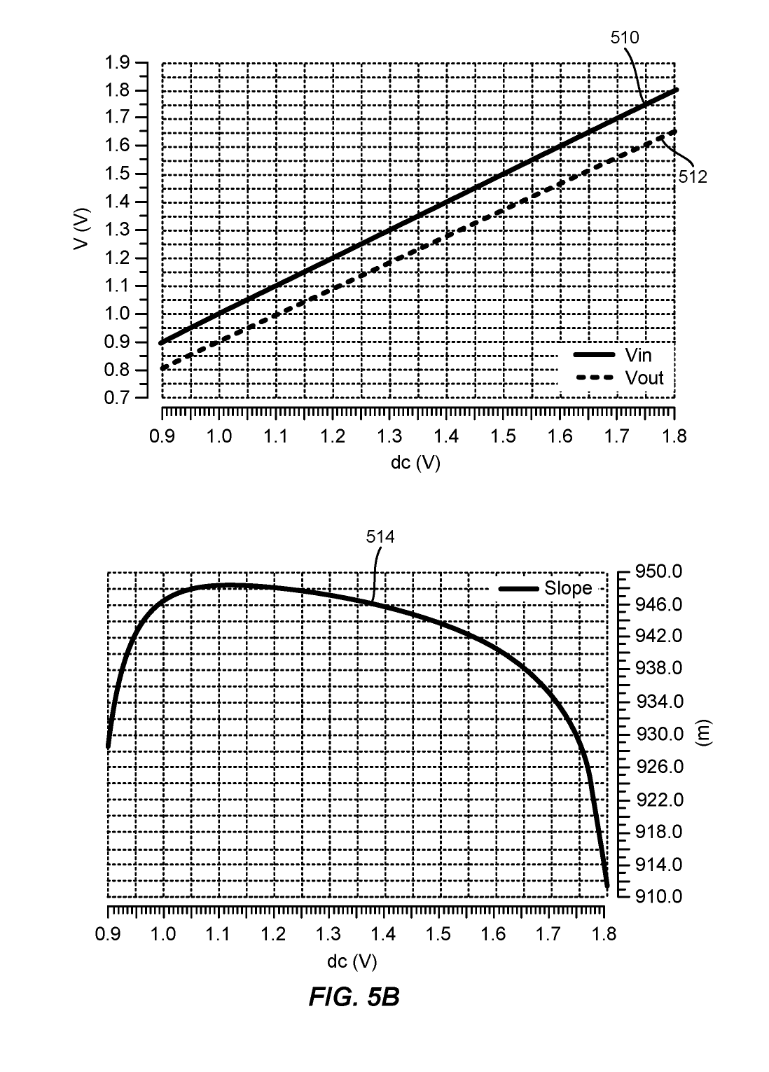

FIG. 5A is a circuit diagram illustrating yet another example sensing cell 500, in accordance with an aspect of the present disclosure. Sensing cell 500 is similar to sensing cell 300 of FIG. 3A, where like numerals and like labels are used to refer to like elements. However, the illustrated example of sensing cell 500 includes a voltage buffer 502 implemented as a two transistor and two current source device. In particular, the illustrated example of voltage buffer 502 includes a first transistor t1, a second transistor t2, a first current source I1, and a second current source I2. In one example, voltage buffer 502 consists of the first transistor t1, the second transistor t2, the first current source I1, and the second current source I2. That is, in some implementations the illustrated example of voltage buffer 502 may include no additional transistors beyond transistors t1 and t2 (other than transistors that may be included in the current sources I1 and I2) and may include no additional current sources other than current sources I1 and I2. Furthermore, the illustrated example of voltage buffer 502 does not include an operational amplifier (op-amp). Sensing cell 500 of FIG. 5A illustrates one possible circuitry architecture for implementing each sensing cell within array 205 of FIG. 2.

As shown in FIG. 5A, the voltage buffer 502 is coupled between the integrating capacitor C1 and the column readout line to provide the buffered voltage V_2 to the column readout line. In particular, the voltage buffer 502 includes an input 504 coupled to receive the voltage V_1 stored on the integrating capacitor and an output 506 coupled to provide the buffered voltage V_2 to the column readout line. As shown, the first transistor t1 includes a gate G coupled to the input 504 to receive the voltage V_1, a drain D coupled to the supply voltage VDD, and a source S coupled to the first current source I1. The second transistor t2 is shown as including a gate G coupled to output 506 to provide the buffered voltage V_2 to the column readout line, a drain D coupled to the gate G and to the second current source I2, and a source S coupled to the first current source I1. The first current source I1 is coupled between the sources of the first and second transistors and the common reference 310. The second current source I2 is coupled to the supply voltage VDD between the supply voltage VDD and the drain D of the second transistor t2.

In one example, the above-referenced couplings of the first transistor t1, second transistor t2, first current source I1, and second current source I2 are direct couplings. That is, a direct coupling may refer to a connection without any intervening active components there between. For example, the source S of transistor t1 may be directly coupled to the first current source I1 without any intervening active components (e.g., transistor, diode, integrated circuit, etc.) between the source S and the first current source I1. In some implementations, a direct coupling without an intervening active component may still include an intervening passive component, in accordance with the teachings herein. That is, a passive component (e.g., a resistor) may be connected in between the gate G of the second transistor t2 and the output 506 such that a current or voltage is still allowed to propagate from the gate G to the output 506. In yet another example, the above-referenced couplings may refer to a direct connection without any intervening active or passive components there between. For example, the gate G of the first transistor t1 may be directly connected to the integrating capacitor C1 without any intervening active components (e.g., transistor, diode, etc.) and without any intervening passive components (e.g., resistor, capacitor, etc.). However, a direct connection between the illustrated components of voltage buffer 502 may still include a mechanism for passing signals there between such as a conductor, metal trace, via, lead, wire, etc.

In one aspect, the first transistor t1 and the first current source I1 are configured to operate as a source follower similar to the source follower configuration of FIG. 3A. Thus, as the stored voltage V_1 is applied to the gate G of the first transistor t1, the voltage at the source S of the first transistor T1 also increases. However, the rate of increase in voltage at the source S of first transistor T1 is less than the rate of increase in voltage at the gate G of the first transistor T1, due in part to the body effect of the first transistor t1. As mentioned above, the body effect of a source follower transistor may affect the resultant gain. In one example, the gain between the gate G and source S of the first transistor t1 may be about 0.8. Thus, voltage buffer 502 includes the addition of the second transistor t2 and second current source I2 configured to compensate for this body effect of the first transistor t1. For example, as shown in FIG. 5A, the second transistor t2 is coupled to accept its input at the source S of the second transistor T2. This configuration may provide for at least some mitigation, if not a complete reversal of the effect that the first transistor t1's body effect has on the overall gain and/or linearity of the voltage buffer 502. In one example, the addition of the second transistor T2 and second current source I2 results in a recovery of the overall gain of voltage buffer 502 to around 0.95. To be sure, FIG. 5B is a diagram illustrating a linearity and a gain performance of an example implementation of the sensing cell 500 of FIG. 5A. As shown in FIG. 5B, the output voltage 512 (i.e., buffered voltage V_2) of the voltage buffer 502 exhibits improved linearity with respect to the input voltage 510 (i.e., voltage V_1). Also, the gain 514 of the voltage buffer 502 is in the range of about 0.942-0.950, which is an increase when compared to the gain 312 of FIG. 3B.

In one example, the overall gain of the voltage buffer 502 is proportional to the intrinsic resistance of the second current source I2. In particular, the gain of voltage buffer 502 may be expressed as

$$\frac{V\_ 2}{V\_ 1} = \frac{{g}_{m} + {g}_{ds} + {g}_{mb}}{\frac{1}{{R}_{s}} + {g}_{m} + {g}_{ds}} ,$$

where gₘ is the transconductance of the second transistor t2, g_{mb} is the gain due to the body effect of the second transistor t2, g_{ds} is the drain-to-source gain of the second transistor t2, and Rₛ is the intrinsic resistance of the second current source I2.

Although FIG. 5A illustrates the first transistor t1 and the second transistor t1 as field effect transistors (FETs), other transistor types may be implemented for one or more of the illustrated transistors, including, but not limited to bipolar junction transistors (BJTs). Furthermore, although FIG. 5A illustrates the first transistor t1 and the second transistor t1 as N-type metal oxide semiconductor (NMOS) transistors, other conductivity types may be implemented for one or more of the illustrated examples, such as P-type metal oxide semiconductor (PMOS) transistors.

In some implementations, the voltage buffer 502 is configured such that the voltage at the gate G of the first transistor t1 is substantially equal to the voltage at the gate G of the second transistor t2. However, as mentioned above, the second current source I2 may include an intrinsic resistance that results in a voltage drop across the second current source I2. Thus, in some examples the relative sizing of the first transistor t1 to the second transistor t2 may be configured to adjust for this. In one example, the size of the second transistor t2 may be larger than the size of the first transistor t1. In some aspects, the size of the transistor may refer to the channel width. Thus, a larger size refers to a larger channel width. In another example, the size of the transistor refers to a ratio of the transistors channel width to channel length (e.g., channel width/channel length). Thus, in this example, a larger size refers to a transistor that has a larger ratio of channel width to channel length.

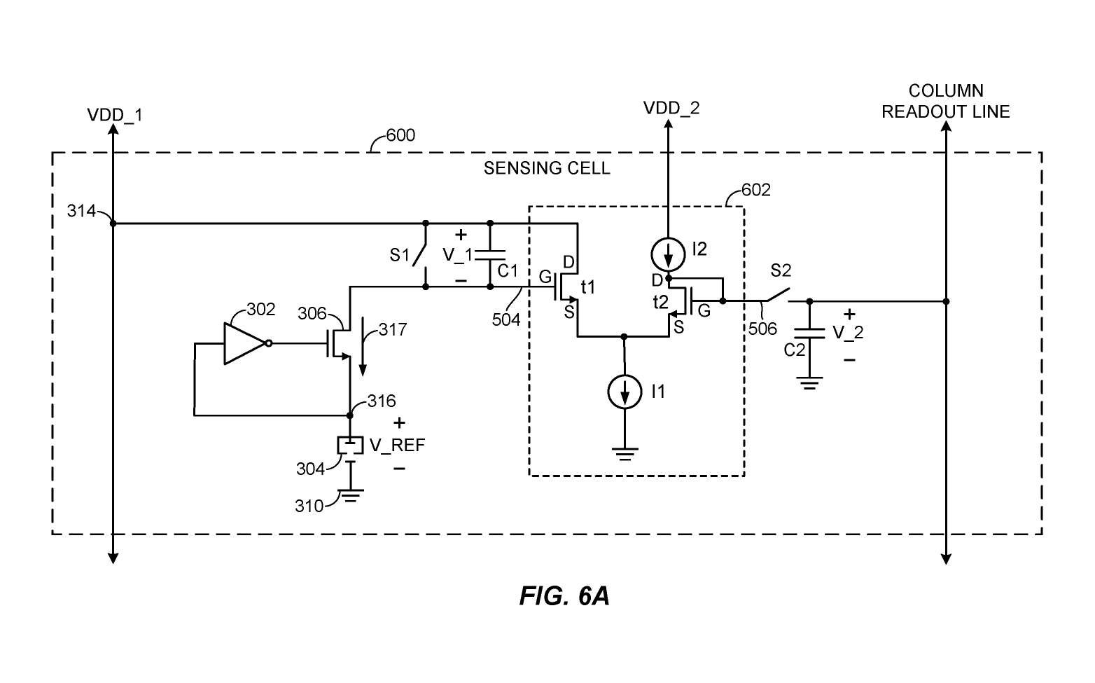

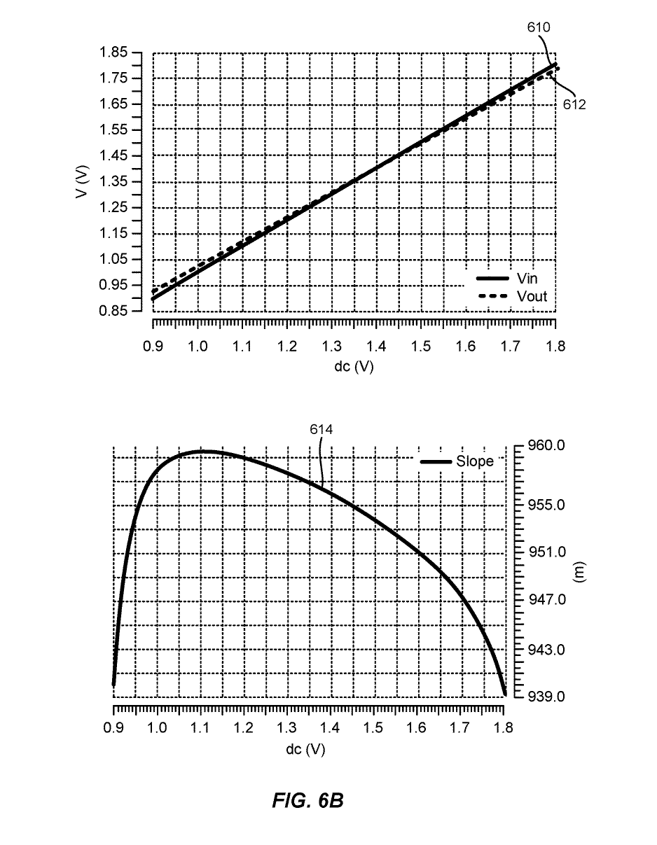

In lieu of, or in addition to, having a size of the first transistor t1 different than the size of the second transistor t2, the voltage buffer 502 may be configured to utilize two separate supply voltages to compensate for the voltage drop across the second current source I2. By way of example, FIG. 6A is a circuit diagram illustrating an example sensing cell 600 that includes a voltage buffer 602 that is coupled to two supply voltages (i.e., first supply voltage VDD_1 and second supply voltage VDD_2), in accordance with an aspect of the present disclosure. Sensing cell 600 is similar to sensing cell 500 of FIG. 5A, where like numerals and like labels are used to refer to like elements. Sensing cell 600 of FIG. 6A illustrates one possible circuitry architecture for implementing each sensing cell within array 205 of FIG. 2.

As shown in FIG. 6A, the voltage buffer 602 is coupled to a first supply voltage VDD_1 and also to a second supply voltage VDD_2. In one aspect, a value of the voltage provided by the second supply voltage VDD_2 is greater than a value of the voltage provided by the first supply voltage VDD_1 (i.e., VDD_2>VDD_1). In one example, the value of the voltage provided by the second supply voltage VDD_2 is selected to provide a sufficient drain-to-source voltage V_{ds} across the drain D to source S of the second transistor t2 considering the voltage drop across the second current source I2. In some implementations the value of the voltage provided by the first supply voltage VDD_1 may be about 1.8V, and the value of the voltage provided by the second supply voltage VDD_2 may be about 2.0V.

As shown above, the voltage drop across the second current source I2 may be compensated for to ensure a sufficient V_{ds} across the second transistor t2 by utilizing separate supply voltages for the voltage buffer 602. In one example, the values of the first and second supply voltages are selected such that the size of the first transistor t1 can be the same as the size of the second transistor t2. In yet another example, the values of the first and second supply voltages are selected such that the size of the second transistor t2 is smaller than a size of the first transistor t1. That is, in the illustrated example of the voltage buffer 602 of FIG. 6A, the value of the voltage provided by the second supply voltage VDD_2 may be selected such that not only is the voltage drop across the current source I2 compensated for, but the value of the voltage provided is further increased to allow the size of the second transistor t2 to be less than the size of the first transistor t1. Thus, in this example, the physical area occupied by the sensing cell 600 may be further reduced by the reduction in the size of the second transistor t2.

FIG. 6B is a diagram illustrating a linearity and a gain performance of an example implementation of the sensing cell 600 of FIG. 6A. As shown in FIG. 6B, the output voltage 612 (i.e., buffered voltage V_2) of the voltage buffer 602 exhibits improved linearity with respect to the input voltage 610 (i.e., voltage V_1). Also, the gain 614 of the voltage buffer 602 is in the range of about 0.955-0.960, which is an increase when compared to the gain 312 of FIG. 3B.

Thus, the example circuitry architectures of voltage buffers 502 and 602 provide for an improvement in linearity and gain when compared to the circuitry architecture of the voltage buffer 308 of FIG. 3A. Furthermore, the example circuitry architectures of voltage buffers 502 and 602 consume less area (i.e., occupy less area on the circuit die) when compared to the circuitry architecture of the voltage buffer 402 of FIG. 4A.

Those of skill in the art will appreciate that information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

Further, those of skill in the art will appreciate that the various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure. By way of example, for each of the embodiments described herein, the corresponding form of any such embodiments may be described herein as, a “means for” performing the described action. Thus, a “means for generating a current signal” may correspond to, for example, ionic current sensor 102 of FIG. 1, current sensor 304 of FIGS. 3A, 4A, 5A, and 6A, and/or to any general current sensor for sensing a small signal current. Similarly, a “means for storing a voltage representative of the current signal” may correspond to, for example, integrating capacitor C1 of FIGS. 3A, 4A, 5A, and 6A.

The methods, sequences and/or algorithms described in connection with the embodiments disclosed herein may be embodied directly in hardware, in a software module executed by a processor, or in a combination of the two. A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor such that the processor can read information from, and write information to, the storage medium. In the alternative, the storage medium may be integral to the processor.

Accordingly, an aspect of the present disclosure can include a computer readable media embodying a method for reading out one or more current values from a current sensor array. Accordingly, aspects of the present disclosure are not limited to illustrated examples and any means for performing the functionality described herein are encompassed by the scope of the present disclosure.

While the foregoing disclosure shows illustrative embodiments, it should be noted that various changes and modifications could be made herein without departing from the scope of the present disclosure as defined by the appended claims. The functions, steps and/or actions of the method claims in accordance with examples described herein need not be performed in any particular order. Furthermore, although elements of the disclosed examples may be described or claimed in the singular, the plural is contemplated unless limitation to the singular is explicitly stated.

What is claimed is:

1. A sensing cell, comprising:
a current sensor configured to generate a current signal;
an integrating capacitor configured to store a voltage representative of the current signal; and
a voltage buffer coupled between the integrating capacitor and a readout line to provide a buffered voltage to the readout line, wherein the voltage buffer comprises:
a first transistor coupled to receive the voltage stored on the integrating capacitor; and
a second transistor coupled to the readout line and configured to compensate for a body effect of the first transistor.

2. The sensing cell of claim 1, wherein a size of the second transistor is larger than a size of the first transistor.

3. The sensing cell of claim 1, further comprising a sense transistor coupled between the integrating capacitor and the current sensor to provide the current signal to the integrating capacitor.

4. The sensing cell of claim 1, wherein the voltage buffer further comprises:
a first current source coupled to the first transistor and to the second transistor; and
a second current source coupled to the second transistor.

5. The sensing cell of claim 4, wherein,
a gate of the first transistor is coupled to the integrating capacitor;
a drain of the first transistor is coupled to a first supply voltage;
a source of the first transistor is coupled to the first current source;
the second current source is coupled between the first supply voltage and a drain of the second transistor;
the drain of the second transistor is coupled to a gate of the second transistor;
a source of the second transistor is coupled to the first current source; and
the gate of the second transistor is coupled to provide the buffered voltage to the readout line.

6. The sensing cell of claim 4, wherein the voltage buffer is coupled to a first supply voltage and to a second supply voltage.

7. The sensing cell of claim 6, wherein a value of the voltage provided by the second supply voltage is greater than a value of the voltage provided by the first supply voltage.

8. The sensing cell of claim 7, wherein a size of the second transistor is the same as a size of the first transistor.

9. The sensing cell of claim 7, wherein a size of the second transistor is smaller than a size of the first transistor.

10. The sensing cell of claim 7, wherein:
a gate of the first transistor is coupled to the integrating capacitor;
a drain of the first transistor is coupled to the first supply voltage;
a source of the first transistor is coupled to the first current source;
the second current source is coupled between the second supply voltage and a drain of the second transistor;
the drain of the second transistor is coupled to a gate of the second transistor;
a source of the second transistor is coupled to the first current source; and
the gate of the second transistor is coupled to provide the buffered voltage to the readout line.

11. The sensing cell of claim 4, wherein the voltage buffer consists of the first transistor, the second transistor, the first current source, and the second current source.

12. The sensing cell of claim 1, wherein the current sensor comprises an ionic current sensor.

13. The sensing cell of claim 1, wherein the voltage buffer comprises an input coupled to receive the voltage stored on the integrating capacitor and an output coupled to provide the buffered voltage to the readout line, and wherein,
a gate of the first transistor is coupled to the input of the voltage buffer; and
a gate of the second transistor is coupled to a drain of the second transistor and to the output of the voltage buffer.

14. A sensing cell, comprising:
means for generating a current signal;
means for storing a voltage representative of the current signal;
a voltage buffer coupled to the means for storing the voltage and configured to generate a buffered voltage, wherein the voltage buffer comprises:
a first transistor coupled to receive the voltage representative of the current signal; and
a second transistor coupled to generate the buffered voltage and configured to compensate for a body effect of the first transistor.

15. The sensing cell of claim 14, wherein the voltage buffer further comprises:
a first current source coupled to the first transistor and to the second transistor; and
a second current source coupled to the second transistor.

16. The sensing cell of claim 15, wherein,
a gate of the first transistor is coupled to receive the voltage representative of the current signal;
a drain of the first transistor is coupled to a first supply voltage;
a source of the first transistor is coupled to the first current source;
the second current source is coupled between the first supply voltage and a drain of the second transistor;
the drain of the second transistor is coupled to a gate of the second transistor;
a source of the second transistor is coupled to the first current source; and
the gate of the second transistor is coupled to generate the buffered voltage.

17. The sensing cell of claim 15, wherein the voltage buffer is coupled to a first supply voltage and to a second supply voltage and wherein a value of the voltage provided by the second supply voltage is greater than a value of the voltage provided by the first supply voltage.

18. The sensing cell of claim 17, wherein:
a gate of the first transistor is coupled to receive the voltage representative of the current signal;
a drain of the first transistor is coupled to the first supply voltage;
a source of the first transistor is coupled to the first current source;
the second current source is coupled between the second supply voltage and a drain of the second transistor;
the drain of the second transistor is coupled to a gate of the second transistor;
a source of the second transistor is coupled to the first current source; and
the gate of the second transistor is coupled to generate the buffered voltage.

19. The sensing cell of claim 15, wherein the voltage buffer consists of the first transistor, the second transistor, the first current source, and the second current source.

20. A complementary metal-oxide-semiconductor (CMOS) ionic current sensor array, comprising:
a plurality of sensing cells, wherein each sensing cell of the plurality of sensing cells includes:
an ionic current sensor configured to generate a current signal;
an integrating capacitor configured to store a voltage representative of the current signal;
a sense field effect transistor (FET) coupled between the integrating capacitor and the current sensor to provide the current signal to the integrating capacitor; and
a voltage buffer coupled between the integrating capacitor and a column readout line to provide a buffered voltage to the column readout line, wherein the voltage buffer comprises:
a first FET coupled to receive the voltage stored on the integrating capacitor;
a second FET coupled to the column readout line and configured to compensate for a body effect of the first FET;
a first current source coupled to the first FET and to the second FET; and
a second current source coupled to the second FET.
